(19) United States
(12) Patent Application Publication
(10) Pub. No.: US 2018/0273507 A1
(43) Pub. Date: Sep. 27, 2018

(54) CRYSTAL FORMS OF A M1 RECEPTOR POSITIVE ALLOSTERIC MODULATOR

(71) Applicant: Merck Sharp & Dohme Corp., Rahway, NJ (US)

(72) Inventors: C. Scott Shultz, Maplewood, NJ (US); Courtney Maguire, Piscataway, NJ (US); Yanke Xu, Bridgewater, NJ (US)

(73) Assignee: Merck Sharp & Dohme Corp., Rahway, NJ (US)

(21) Appl. No.: 15/762,031

(22) PCT Filed: Sep. 26, 2016
(86) PCT No.: PCT/US16/53649

§ 371 Date: Mar. 21, 2018

Related U.S. Application Data
(60) Provisional application No. 62/235,073, filed on Sep. 30, 2015.

(51) Int. Cl. C07D 401/06; C07C 57/13

(52) U.S. Cl. CPC C07D 401/06; C07B 2200/13; C07C 57/13

(57) ABSTRACT

The invention is directed to novel anhydrous crystalline butenedioate salt form 1 of Compound I, which is a modulator of muscarinic M1 receptors. The novel crystalline forms of compound I are useful in the treatment or prevention of Alzheimer's disease and other disorders and diseases in which muscarinic M1 receptors are involved. The invention is further directed to pharmaceutical compositions comprising the novel crystalline forms of Compound I.

### BACKGROUND OF THE INVENTION

Alzheimer's disease is a common neurodegenerative disease affecting the elderly, resulting in progressive memory impairment, loss of language and visuospatial skills, and behavior deficits. Characteristics of the disease include degeneration of cholinergic neurons in the cerebral cortex, hippocampus, basal forebrain, and other regions of the brain, neurofibrillary tangles, and accumulation of the amyloid β peptide (Aβ). Aβ is a 39-43 amino acid produced in the brain by processing of the beta-amyloid precursor protein (APP) by the beta-amyloid protein cleaving enzyme (“beta secretase” or “BACE”) and gamma-secretase. The processing leads to accumulation of Aβ in the brain.

Cholinergic neurotransmission involves the binding of acetylcholine either to the nicotinic acetylcholine receptor (nAChR) or to the muscarinic acetylcholine receptor (mAChR). It has been hypothesized that cholinergic hypofunction contributes to the cognitive deficits of patients suffering from Alzheimer's Disease. Consequently, pharmacotherapeutic targets which increase the activation of muscarinic receptors to counteract cholinergic hypofunction have been explored. Muscarinic receptors are prevalent throughout the body. Five distinct muscarinic receptors (M1-M5) have been identified in mammals. In the central nervous system, muscarinic receptors are involved in cognitive, behavior, sensory, motor and autonomic functions. The muscarinic M1 receptor, which is prevalent in the cerebral cortex, hippocampus and striatum, has been found to have a major role in cognitive processing and is believed to have a role in the pathophysiology of Alzheimer's Disease. See Eglen et al, TRENDS in Pharmacological Sciences, 2001, 22:8, 409-414. Additionally, M1 agonists also have the potential to treat the underlying disease mechanism of Alzheimer's Disease. The cholinergic hypothesis of Alzheimer's Disease is linked to both β-amyloid and hyperphosphorylated tau protein. Formation of β-amyloid may impair the coupling of the muscarinic receptor with G-proteins. Stimulation of the M1 muscarinic receptor has been shown to increase formation of the neuroprotective aAPPs fragment, thereby preventing the formation of the Aβ peptide. Thus, M1 agonists may alter APP processing and enhance αAPPs secretion. See Fisher, Jpn J Pharmacol, 2000, 84:101-112.

The compound of the Formula I:

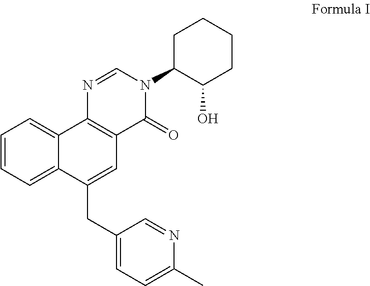 [SMILES: Cc1ccc(Cc2cc3c(=O)n([C@H]4CCCC[C@@H]4O)cnc3c3ccccc23)cn1]

is disclosed as a modulator of muscarinic M1 receptors in U.S. Pat. No. 8,557,832, and PCT Patent Publication WO 2010/059773 incorporated herein in its entirety. As described in the cited references, compound I may be synthesized as a free base or salt. Example 2 of WO2010/059773 describes a medicinal chemistry route for making a free base of compound of Formula I.

The physical and biological attributes of a drug's active ingredient, such as solubility, stability, melting point, bioavailability, and the like can be affected by the solid-state form. There remains a need in the art to identify a suitable solid-state form of compound I which may be beneficial to achieving acceptable biological and physical properties while minimizing difficulties with drug substance manufacturing, processing, and storage. The present invention relates to this need.

### SUMMARY OF THE INVENTION

The invention is directed to two novel crystalline forms of the compound of Formula I which are described below. The pharmaceutically advantageous crystalline forms of the invention are useful in the treatment of Alzheimer's Disease and other diseases mediated by the muscarinic M1 receptor. The invention is further directed to pharmaceutical compositions comprising the pharmaceutically advantageous crystalline forms of Formula I in the treatment of Alzheimer's Disease and other diseases (e.g., cognitive impairment, schizophrenia, pain disorders, and sleep disorders) mediated by the muscarinic M1 receptor. In a particular aspect, the invention is directed to crystalline Compound I free base, form 1 of the formula:

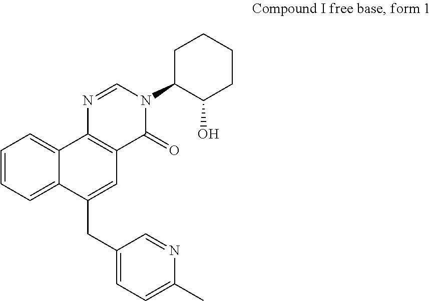 [SMILES: Cc1ccc(Cc2cc3c(=O)n([C@H]4CCCC[C@@H]4O)cnc3c3ccccc23)cn1]

and as further described herein.

In a particular aspect, the invention is directed to crystalline Compound I butenedioate salt, form 1 of the formula:

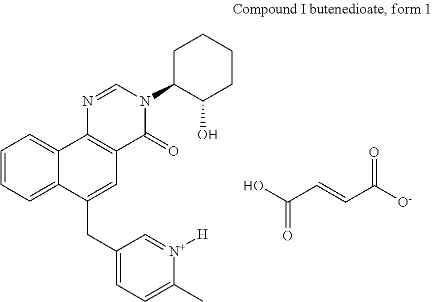 [SMILES: Cc1ccc(Cc2cc3c(=O)n([C@H]4CCCC[C@@H]4O)cnc3c3ccccc23)c[nH+]1 ; O=C([O-])/C=C/C(=O)O]

and as further described herein.

### BRIEF DESCRIPTION OF THE DRAWINGS

FIG. 1 shows the X-ray powder diffraction pattern for the anhydrous crystalline compound I butenedioate form 1.

FIG. 2 shows a DSC thermogram for anhydrous crystalline Compound I butenedioate form 1.

FIG. 3 shows a TGA thermogram for anhydrous crystalline Compound I butenedioate form 1.

FIG. 4 shows the X-ray powder diffraction pattern for anhydrous crystalline Compound I freebase form 1.

FIG. 5 shows a DSC thermogram for anhydrous crystalline Compound I freebase form 1.

FIG. 6 shows a TGA thermogram for anhydrous crystalline Compound I freebase form 1.

FIG. 7 shows solid-state carbon-13 nuclear magnetic resonance (NMR) spectrum for anhydrous crystalline Compound I free base form 1.

FIG. 8 shows solid-state carbon-13 nuclear magnetic resonance (NMR) spectrum for anhydrous crystalline Compound I butenedioate form 1.

### DETAILED DESCRIPTION OF THE INVENTION

Formula I free base may be referred to, for example, as “3-[(1S,2S)-2-hydroxycyclohexyl]-6-[(6-methylpyridin-3-yl)methyl]benzo[h]quinazolin-4(3H)-one,”. Crystalline Compound I free base may be referred to, for example, as crystalline “3-[(1S,2S)-2-hydroxycyclohexyl]-6-[(6-methylpyridin-3-yl)methyl]benzo[h]quinazolin-4(3H)-one,” Crystalline butenedioate form 1 of Compound I may be referred to, for example as as crystalline “3-[(1S,2S)-2-Hydroxycyclohexyl]-6-[(6-methyl-3-pyridinyl)methyl]benzo-[h]quinazolin-4(3H)-one (2E)-2-butenedioate” salt; or crystalline “3-[(1S,2S)-2-Hydroxycyclohexyl]-6-[(6-methyl-3-pyridinyl)methyl]-benzo[h]quinazolin-4(3H)-one (2E)-but-2-enedioate” salt. It is understood herein that (2E)-but-2-enedioate or (2E)-2-butenedioate is equivalent to fumarate salt. It is also understood that the term butenedioate is equivalent to (2E)-but-2-enedioate, or (2E)-2-butenedioate.

The invention is directed to a pharmaceutically advantageous crystalline forms of Formula I. An embodiment of the invention is directed to a crystalline form 1 of butenedioate salt of Compound I. In an alternate embodiment, the invention is directed to an anhydrous crystalline of butenedioate salt of Compound I. In an alternate embodiment, the invention is directed to an isolated anhydrous crystalline butenedioate salt form 1 of Compound I.

In an alternate embodiment, the invention is directed to a crystalline Compound I free base form 1. In an alternate embodiment, the invention is directed to an anhydrous crystalline compound I free base form 1. In an alternate embodiment, the invention is directed to an isolated anhydrous crystalline Compound I free base form 1.

In an alternate embodiment, the invention is directed to a pharmaceutical composition that comprises crystalline Compound I butenedioate salt, form 1. In an alternate embodiment, the invention is directed to a pharmaceutical composition that comprises crystalline Compound I butenedioate salt, form 1 which is at least about 40 wt. % of the crystalline Compound I butenedioate salt presented in the composition. In an alternate embodiment, the invention is directed to a pharmaceutical composition that comprises crystalline Compound I butenedioate salt, form 1 which is at least about 50 wt. % of the crystalline Compound I butenedioate salt presented in the composition. In an alternate embodiment, the invention is directed to a pharmaceutical composition that comprises crystalline Compound I butenedioate salt, form 1 which is at least about 60 wt. % of the crystalline Compound I butenedioate salt presented in the composition. In an alternate embodiment, the invention is directed to a pharmaceutical composition that comprises crystalline Compound I butenedioate salt, form 1 which is at least about 70 wt. % of the crystalline Compound I butenedioate salt presented in the composition. In an alternate embodiment, the invention is directed to a pharmaceutical composition that comprises crystalline Compound I butenedioate salt, form 1 which is at least about 80 wt. % of the crystalline Compound I butenedioate salt presented in the composition. In an alternate embodiment, the invention is directed to a pharmaceutical composition that comprises crystalline Compound I butenedioate salt, form 1 which is at least about 90 wt. % of the crystalline Compound I butenedioate salt presented in the composition. In an alternate embodiment, the invention is directed to a pharmaceutical composition that comprises crystalline Compound I butenedioate salt, form 1 which is at least about 95 wt. % of the crystalline Compound I butenedioate salt presented in the composition. In an alternate embodiment, the invention is directed to a pharmaceutical composition that comprises crystalline Compound I butenedioate salt, form 1 which is at least about 98 wt. % of the crystalline Compound I butenedioate salt presented in the composition. In an alternate embodiment, the invention is directed to a pharmaceutical composition that comprises crystalline Compound I butenedioate salt form 1 which is at least about 99 wt. % of the crystalline Compound I butenedioate salt presented in the composition.

In an alternate embodiment, the invention is directed to a pharmaceutical composition that comprises crystalline Compound I free base, form 1. In an alternate embodiment, the invention is directed to a pharmaceutical composition that comprises crystalline Compound I free base, form 1 which is at least about 40 wt. % of the crystalline Compound I free base present in the composition. In an alternate embodiment, the invention is directed to a pharmaceutical composition that comprises crystalline Compound I free base, form 1, which is at least about 50 wt. % of the crystalline Compound I free base present in the composition. In an alternate embodiment, the invention is directed to a pharmaceutical composition that comprises crystalline Compound I free base, form 1 which is at least about 60 wt. % of the crystalline Compound I free base present in the composition. In an alternate embodiment, the invention is directed to a pharmaceutical composition that comprises crystalline Compound I free base, form 1 which is at least about 70 wt. % of the crystalline Compound I free base present in the composition. In an alternate embodiment, the invention is directed to a pharmaceutical composition that comprises crystalline Compound I free base, form 1 which is at least about 80 wt. % of the crystalline Compound I free base present in the composition. In an alternate embodiment, the invention is directed to a pharmaceutical composition that comprises crystalline Compound I free base, form 1 which is at least about 90 wt. % of the crystalline Compound I free base present in the composition. In an alternate embodiment, the invention is directed to a pharmaceutical composition that comprises crystalline Compound I free base, form 1 which is at least about 95 wt. % of the crystalline Compound I free base present in the composition. In an alternate embodiment, the invention is directed to a pharmaceutical composition that comprises crystalline Compound I free base, form 1 which is at least about 98 wt. % of the crystalline Compound I free base present in the composition. In an alternate embodiment, the invention is directed to a pharmaceutical composition that comprises crystalline Compound I free base, form 1 which is at least about 99 wt. % of the crystalline Compound I free base present in the composition.

In an alternate embodiment, the invention is directed to an anhydrous form of the butenedioate salt of Compound I. In an embodiment, the invention is directed to an anhydrous crystalline butenedioate salt form 1 of Compound I.

In an alternate embodiment the present invention provides a novel synthesis of crystalline salt forms of Compound I, e.g. free base, form 1, or butenedioate, form 1. A non-limiting example of this synthesis may be described by the description that follows. Reactants for which a synthesis is not described are available commercially for purchase. An aspect of this embodiment is realized when the compound of Formula I, optionally in the presence of a solvent, is contacted with a suitable acid such as acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methansulfonic, mucic, nitric, pamoic, pantothenic, phosphoric, succinic, sulfuric, tartaric, p-toluenesulfonic acid, and the like. In one embodiment the salt is fumarate, or butenedioate of the compound of Formula I. Still another embodiment of this aspect of the invention is realized when the solvent includes water and/or a suitable organic solvent. Non-limiting examples of organic solvents include EtOH, MeOH, iPrOH, CH₂Cl₂, THF, 2-MeTHF, MTBE, DME (1,2-dimethoxyethane), 1,4-dioxane, CPME (cyclopentyl methyl ether), EtOAc, iPrOAc, tBuOH, t-AmOH, toluene, DMF, DMAc, NMP, and DMSO. In one such embodiment, the organic solvent is EtOH. In a further embodiment of this aspect of the invention, the crystalline salt forming step is conducted at a temperature of about 25° C. to about 85° C. In still a further embodiment, the crystalline salt forming step is conducted by contacting the compound of Formula I with a suitable acid in the presence of an organic solvent at a temperature of about 30° C. to about 75° C., adding crystalline butenedioate Compound I, form I solution as seed and aging the solution for up to 48 hours. In another embodiment, the crystalline salt forming step is conducted by adding over 1 to 24 hours additional acid solution at a temperature of about 25° C. to about 85° C. In yet another embodiment, the crystalline salt forming step is conducted at a temperature of about 40° C. to about 60° C., preferably 45° C. to about 55° C., more preferably 50° C. for 2 to 24 hours. In another embodiment, the crystalline salt forming step is conducted at a temperature of about 45° C. to about 55° C., cooled to about −20° C. to 15° C. over 2 to 12 hours and aged at −20° C. to 15° C. for up to 24 hours. In still another embodiment, the crystalline salt forming step is conducted at a temperature of about 50° C., cooled to about 0° to about 5° C. at a rate of about 2° C. to about 7° C. per hour and aged at about 0° to about 5° C. for up to 24 hours. In yet another embodiment, the crystalline salt forming step is conducted at a temperature of about 50° C., cooled to about 0° to about 5° C. at a rate of about 5° C. per hour and aged at about 0° to about 5° C. for up to 24 hours. The solution can then be filtered and the resultant crystals collected. Alternatively, the resultant crystals can be milled at a temperature of about -10° C. to about 15° C., heated to a temperature of about 25° C. to about 85, preferably about 45° C. to about 55 and then cooled to room temperature before filtration and collection of the crystals

The pharmaceutically advantageous crystalline butenedioate salt, form 1 of Compound I may have benefits relative to other morphological forms of compound I such as greater solubility and/or a faster dissolution rate than crystalline forms of the compound, which may improve bioavailability of the compound, may facilitate a faster onset of therapeutic action, may reduce variability of therapeutic response among subjects, and may reduce any food effect. Crystalline form of the butenedioate salt of compound I exhibits comparable or improved in vivo bioavailability compared with formulations of other forms of compound I. The butenedioate salt of Compound I is absorbed more rapidly after oral administration of the formulation. The AUC of the drug and the maximal concentration of the drug in the blood or serum are increased when the crystalline form of the butenedioate salt of Compound I is administered to a patient.

A particular butenedioate salt of crystalline Compound I is the mono butenedioate salt.

X-ray powder diffraction studies are widely used to characterize molecular structures, crystallinity, and polymorphism. The X-ray powder diffraction patterns referenced herein were generated on a Panalytical X-pert Pro PW3040 System configured in the Bragg-Brentano configuration and equipped with a Cu (copper) radiation source with monochromatization to Kα achieved using a Nickel filter. A fixed slit optical configuration was employed for data acquisition. Data was acquired between 2 and 40° 2θ. Samples were prepared by gently pressing powdered sample onto a shallow cavity zero background silicon holder. Single crystal structure determination was performed using a Bruker APEX 2 CCD diffractometer. Cell determinations and diffraction data were measured using monochromatized Cu Kα radiation. Full data acquisition for structure solution was performed at 173K with unit cell dimensions also acquired at 273K.

In addition to obtaining X-ray powder diffraction patterns, samples were further characterized by Differential Scanning calorimeter (DSC) data using TA Instruments DSC 2920 or equivalent instrumentation. A sample with a weight between 2 and 6 mg was weighed into a pan and the pan was crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min from 25° C. to a temperature of approximately 300° C. When the run was completed, the data were analyzed using the DSC analysis program in the system software. The observed endotherms were integrated between baseline temperature points that are above and below the temperature range over which the endotherm is observed. The data reported are the onset temperature, peak temperature and enthalpy. Thermogravimetric analysis (TGA) was carried out on a TA Q 500 Thermogravimetric Analyzer (TA Instrument). Samples (5-15 mg) in open pans were heated from 25 to 300° C. at 10° C./min, with a nitrogen purge of 100 mL/min.

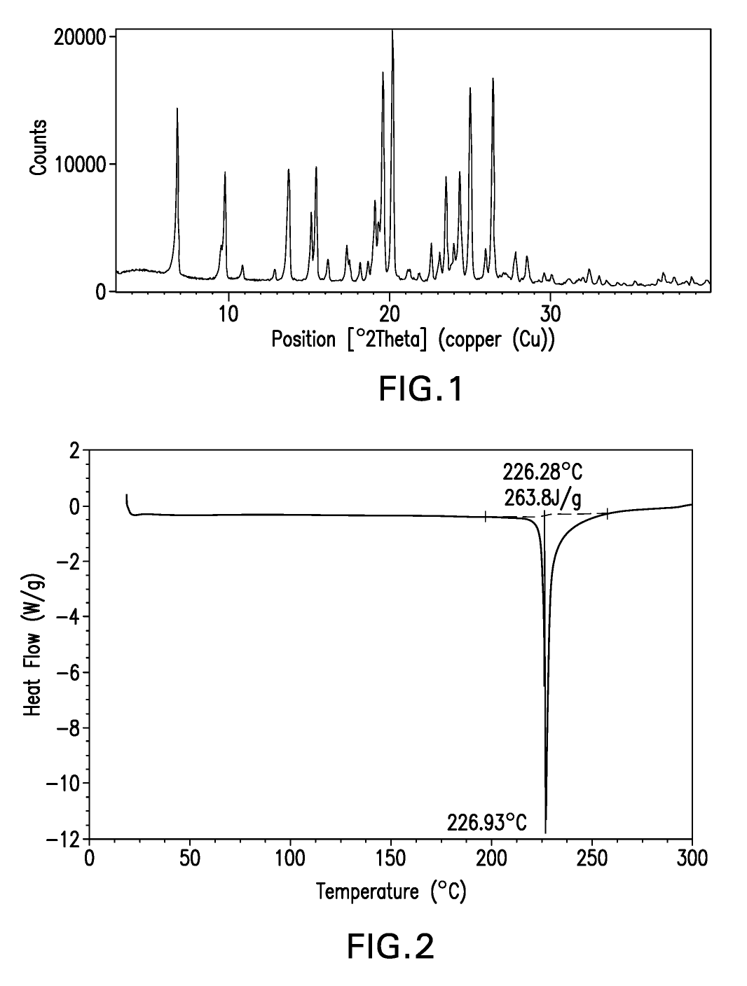

FIG. 1 shows the X-ray powder diffraction pattern for anhydrous crystalline butenedioate salt form 1 of Compound I. The anhydrous crystalline butenedioate salt form 1 is characterized by diffraction peaks corresponding to d-spacings as detailed in Table 1. The anhydrous crystalline butenedioate salt form 1 exhibited characteristic diffraction peaks corresponding to d-spacings as illustrated in Table 1 with preferred diagnostic peak sets 1, 2, and 3 (fourth column). The anhydrous crystalline butenedioate salt form 1 was further characterized by the d-spacings of as illustrated in Table 1 with preferred diagnostic peak sets 1 and 2 (fourth column). The anhydrous crystalline butenedioate salt form 1 was even further characterized by the d-spacings as illustrated in Table 1 with a preferred diagnostic peak set of 1 (fourth column).

TABLE 1
D-spacings of anhydrous crystalline butenedioate salt
form 1 of Compound I as also shown in FIG. 1:

| | | | Preferred |
|---|---|---|---|
| Pos. | d-spacing | Rel. Int. | Diagnostic |
| [°2Th.] | [Å] | [%] * | Peak Set * |
| 4.71 | 18.78 | 2 | 3 |
| 6.90 | 12.81 | 64 | 1 |
| 9.58 | 9.24 | 13 | 2 |
| 9.86 | 8.97 | 40 | 1 |
| 10.94 | 8.09 | 6 | 3 |
| 12.95 | 6.84 | 4 | 3 |
| 13.84 | 6.40 | 39 | 1 |
| 15.19 | 5.83 | 27 | 1 |
| 15.52 | 5.71 | 44 | 1 |
| 16.26 | 5.45 | 8 | 3 |
| 17.42 | 5.09 | 14 | 2 |
| 17.60 | 5.04 | 8 | 3 |
| 18.24 | 4.86 | 7 | 3 |
| 18.75 | 4.73 | 8 | 3 |
| 19.18 | 4.63 | 32 | 1 |
| 19.37 | 4.58 | 24 | 1 |
| 19.64 | 4.52 | 84 | 1 |
| 20.26 | 4.38 | 100 | 1 |
| 21.20 | 4.19 | 5 | 3 |
| 21.33 | 4.17 | 5 | 3 |
| 21.89 | 4.06 | 3 | 3 |
| 22.65 | 3.93 | 15 | 2 |
| 23.19 | 3.84 | 12 | 2 |
| 23.58 | 3.77 | 42 | 1 |
| 24.06 | 3.70 | 16 | 2 |
| 24.42 | 3.64 | 45 | 1 |
| 25.06 | 3.55 | 77 | 1 |
| 26.04 | 3.42 | 13 | 2 |
| 26.48 | 3.37 | 82 | 1 |
| 27.32 | 3.26 | 4 | 3 |
| 27.90 | 3.20 | 12 | 2 |
| 28.58 | 3.12 | 11 | 2 |
| 29.32 | 3.05 | 2 | 3 |
| 29.65 | 3.01 | 4 | 3 |
| 30.13 | 2.97 | 4 | 3 |
| 31.22 | 2.87 | 2 | 3 |
| 31.76 | 2.82 | 2 | 3 |
| 32.09 | 2.79 | 3 | 3 |
| 32.47 | 2.76 | 6 | 3 |
| 33.10 | 2.71 | 3 | 3 |
| 33.54 | 2.67 | 2 | 3 |
| 34.22 | 2.62 | 1 | 3 |
| 34.62 | 2.59 | 1 | 3 |
| 35.28 | 2.54 | 2 | 3 |
| 36.36 | 2.47 | 0 | 3 |
| 36.72 | 2.45 | 2 | 3 |
| 37.07 | 2.43 | 5 | 3 |
| 37.72 | 2.39 | 3 | 3 |
| 38.51 | 2.34 | 2 | 3 |
| 38.82 | 2.32 | 3 | 3 |

A. Single Crystal Crystallographic Information Experimental: Single crystal structure determination was performed using a Bruker APEX 2 CCD diffractometer. Cell determinations and diffraction data were measured using monochromatized Cu Kα radiation. Full data acquisition for structure solution was performed at 173K with unit cell dimensions also acquired at 273K. The crystal structure of Compound I butenedioate salt anhydrous form 1 was solved by single crystal x-ray analysis. Crystallographic details are shown in Table 2:

TABLE 2
Crystallographic Information for Compound
I butenedioate salt anhydrous form 1

| Formula, Formula weight | C₂₅H₂₆N₃O₂•C₄H₃O₄, 515.6 |
|---|---|
| Crystal system, Space group | orthorhombic, P2₁2₁2₁ |
| Cell lengths | a = 7.3953 (2), b = 17.8991 (6), |
| | c = 18.5520 (7) |
| Cell angles | α = 90.00, β = 90.00, γ = 90.00 |
| V(Å³), Z, Z′, D(calc) | 2455.71(14), 4, 1, 1.394 |
| μ(Cu Kα) (mm⁻¹) | 0.809 |
| F(000) | 1088 |
| Crystal size (mm) | 0.41 × 0.06 × 0.05 |
| Temperature (K) | 173 |
| Radiation (Å) | Cu Kα (1.54184) |
| Instrument | Bruker APEXII |
| Resolution (Å⁻³), max theta (°) | 0.84, 66.43 |
| Reflections: (Total, Unique, | 17713, 4799, 3079 |
| 2σ Obsd) | |
| Refined parameters | 349 |
| R, wR₂, S | 0.0675, 0.1699, 1.000 |
| Max. shift/error | 0.00 |
| Max. residual density [e Å⁻³] | 0.308 |
| Flack parameter | −0.1(4) |

FIG. 2 shows the DSC thermogram for anhydrous crystalline butenedioate salt form 1 of Compound I. An endotherm with extrapolated onset (Tₒₙₛₑₜ)=226.3° C. Tₚₑₐₖ=226.9° C., and ΔH=263.8 J/g was observed that is consistent with concurrent melting and decomposition.

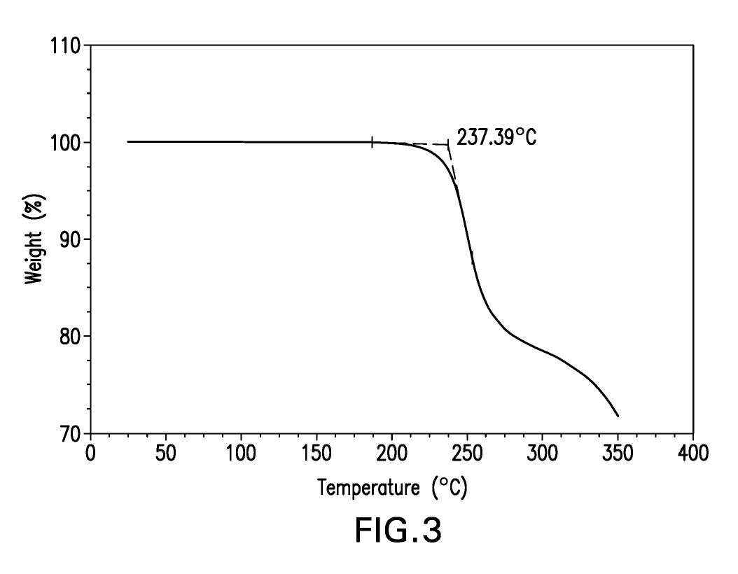

FIG. 3 shows The TGA thermogram for anhydrous crystalline butenedioate salt form 1 of Compound I. No weight loss is observed up until the onset of decomposition at 237.4° C.

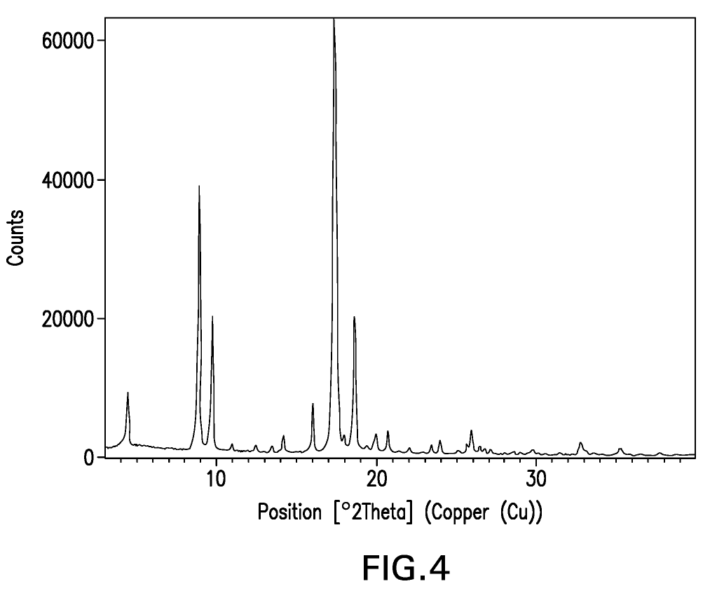

FIG. 4 shows the X-ray powder diffraction pattern for anhydrous crystalline Compound I freebase form 1. The anhydrous crystalline free base form 1 is characterized by diffraction peaks corresponding to d-spacings as detailed in Table 3. The anydrous crystalline free base form 1 of Compound I exhibited characteristic diffraction peaks corresponding to d-spacings in angstroms as illustrated in Table 3 with preferred diagnostic peak sets 1, 2, and 3 (fourth column). The anydrous crystalline free base form 1 of Compound I was further characterized by the d-spacings in angstroms as illustrated in Table 3 with preferred diagnostic peak sets 1 and 2 (fourth column). The anydrous crystalline free base form 1 of Compound I was even further characterized by the d-spacings in angstroms as illustrated in Table 3 with a preferred diagnostic peak set of 1 (fourth column).

TABLE 3
D-spacings of anydrous crystalline free base
form 1 of Compound I as also shown in FIG. 4

| | | | Preferred |
|---|---|---|---|
| Pos. | d-spacing | Rel. Int. | Diagnostic |
| [°2Th.] | [Å] | [%] * | Peak Set * |
| 4.46 | 19.83 | 13 | 1 |
| 8.92 | 9.92 | 60 | 1 |
| 9.73 | 9.09 | 31 | 1 |
| 10.94 | 8.09 | 1 | 3 |
| 12.46 | 7.10 | 1 | 3 |
| 13.45 | 6.58 | 1 | 3 |
| 14.17 | 6.25 | 4 | 2 |
| 16.02 | 5.53 | 11 | 1 |
| 17.33 | 5.12 | 100 | 1 |
| 17.50 | 5.07 | 60 | 1 |
| 17.97 | 4.94 | 4 | 2 |
| 18.62 | 4.77 | 31 | 1 |
| 19.39 | 4.58 | 2 | 2 |
| 19.98 | 4.44 | 4 | 1 |
| 20.70 | 4.29 | 5 | 1 |
| 21.38 | 4.16 | 0 | 3 |
| 22.06 | 4.03 | 1 | 2 |
| 22.88 | 3.89 | 0 | 3 |
| 23.43 | 3.80 | 2 | 2 |
| 23.97 | 3.71 | 3 | 2 |
| 25.06 | 3.55 | 1 | 3 |
| 25.62 | 3.48 | 2 | 2 |
| 25.94 | 3.43 | 6 | 1 |
| 26.46 | 3.37 | 2 | 2 |
| 26.74 | 3.33 | 1 | 2 |
| 27.12 | 3.29 | 1 | 3 |
| 27.96 | 3.19 | 0 | 3 |
| 28.60 | 3.12 | 1 | 3 |
| 28.99 | 3.08 | 0 | 3 |
| 29.75 | 3.00 | 1 | 2 |
| 30.12 | 2.97 | 0 | 3 |
| 30.68 | 2.91 | 0 | 3 |
| 31.41 | 2.85 | 0 | 3 |
| 32.77 | 2.73 | 3 | 2 |
| 33.52 | 2.67 | 1 | 3 |
| 35.22 | 2.55 | 1 | 2 |
| 35.85 | 2.50 | 0 | 3 |
| 36.47 | 2.46 | 0 | 3 |
| 37.61 | 2.39 | 0 | 3 |
| 38.71 | 2.33 | 0 | 3 |

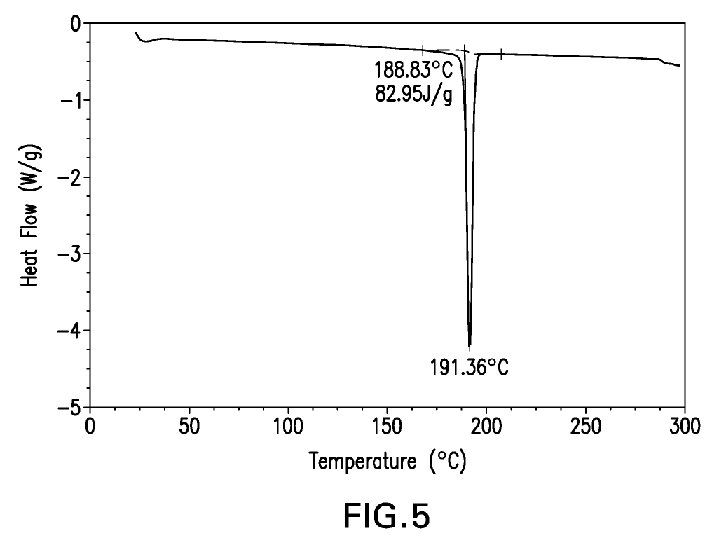

FIG. 5 shows DSC thermogram for anydrous crystalline free base form 1 of Compound I. An endotherm with extrapolated onset (Tₒₙₛₑₜ)=188.8° C. Tₚₑₐₖ=191.4° C., and ΔH=83.0 J/g was observed that is consistent with a melt of the crystalline form.

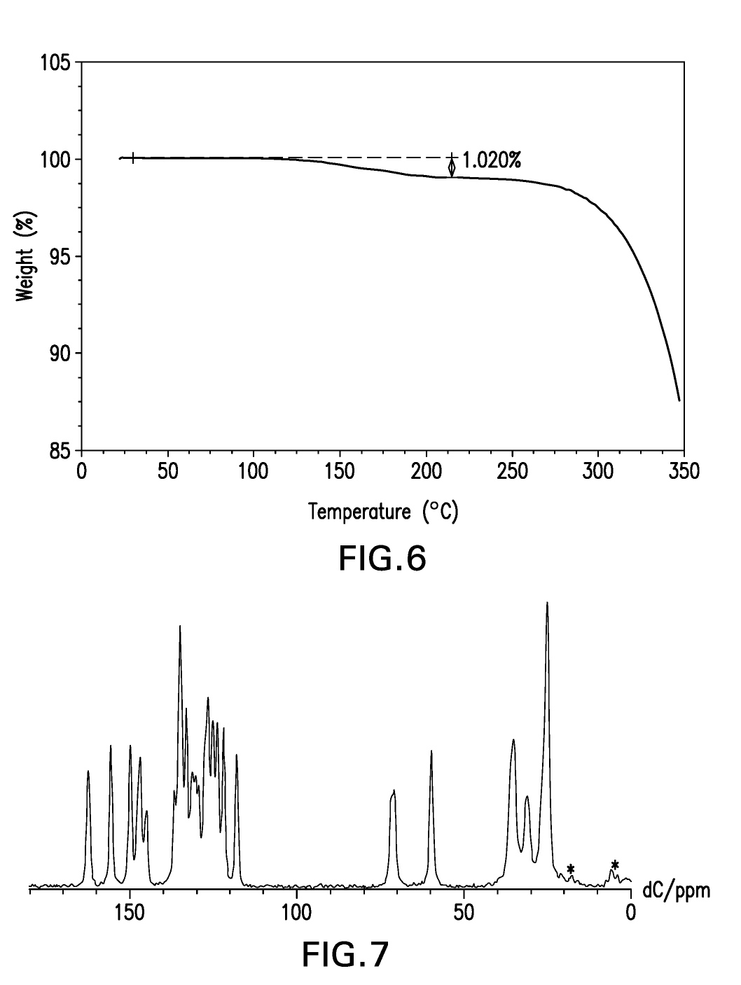

FIG. 6 shows TGA thermogram for anydrous crystalline free base form 1 of Compound I displays a weight loss of 1.02 wt % up until the 215° C., associated with loss of residual solvent upon melting.

Samples were also characterized based on solid-state carbon-13 nuclear magnetic resonance (NMR) spectrum. The carbon-13 spectrum was recorded on a Bruker AV400 NMR spectrometer using a Bruker 4 mm H/F/X BB double resonance CPMAS probe. The spectrum was collected utilizing proton/carbon-13 variable-amplitude cross-polarization (VACP) at 83.3 kHz, with a contact time of 3 ms. Other experimental parameters used for data acquisition were a proton 90-degree pulse of 100 kHz, TPPM decoupling at 100 kHz, a pulse delay of 120 s, and signal averaging for 320 acquisitions. The magic-angle spinning (MAS) rate was set to 13 kHz. A Lorentzian line broadening of 30 Hz was applied to the spectrum before Fourier Transformation. Chemical shifts are reported on the TMS scale using the carbonyl carbon of glycine (176.70 ppm) as a secondary reference.

FIG. 7 shows Solid state carbon-13 CPMAS NMR spectrum of anydrous crystalline free base form 1 of Compound I. Spinning sidebands are indicted by asterisks.

TABLE 4
Characteristic carbon-13 chemical shifts and the corresponding
relative intensities observed for anydrous crystalline
free base form 1 of Compound I:

| Chemical Shifts | Relative Peak |
|---|---|
| [ppm] | Intensity |
| 162.47 | 0.41 |
| 155.75 | 0.50 |
| 149.93 | 0.49 |
| 147.01 | 0.45 |
| 145.22 | 0.26 |
| 136.67 | 0.34 |
| 135.10 | 0.92 |
| 133.19 | 0.63 |
| 131.26 | 0.40 |
| 130.41 | 0.39 |
| 129.52 | 0.35 |
| 126.63 | 0.67 |
| 125.20 | 0.58 |
| 123.87 | 0.58 |
| 121.98 | 0.56 |
| 118.08 | 0.46 |
| 70.96 | 0.34 |
| 59.69 | 0.48 |
| 35.09 | 0.52 |
| 30.98 | 0.32 |
| 25.04 | 1.00 |

Relative peak intensities are normalized to the highest peak. Peak positions and relative intensities were measured with the Bruker TS2.1 software.

FIG. 8 shows Solid state carbon-13 CPMAS NMR spectrum of anhydrous crystalline butenedioate form 1 of Compound I. Spinning sidebands are indicted by asterisks.

TABLE 5
Characteristic carbon-13 chemical shifts and the corresponding
relative intensities observed for anhydrous crystalline
butenedioate form 1 of Compound I:

| Chemical Shifts | Relative Peak |
|---|---|
| [ppm] | Intensity |
| 173.66 | 0.57 |
| 172.70 | 0.61 |
| 160.16 | 0.39 |
| 151.97 | 0.44 |
| 147.52 | 0.52 |
| 146.79 | 0.54 |
| 143.66 | 0.37 |
| 142.78 | 0.51 |
| 140.71 | 0.46 |
| 139.01 | 0.55 |
| 135.03 | 0.45 |
| 131.98 | 0.54 |
| 130.79 | 0.87 |
| 124.83 | 1.00 |
| 119.62 | 0.47 |
| 116.74 | 0.41 |
| 69.74 | 0.48 |
| 68.63 | 0.56 |
| 36.89 | 0.37 |
| 35.44 | 0.46 |
| 29.44 | 0.34 |
| 26.60 | 0.38 |
| 25.32 | 0.40 |
| 19.11 | 0.59 |

Relative peak intensities are normalized to the highest peak. Peak positions and relative intensities were measured with the Bruker TS2.1 software.

The crystalline forms according to the invention have, unexpectedly and beneficially been found to qualify as a preferred BCS (Biopharmaceutics Classification System) Class 1 substance. Additionally, they have each been found to exhibit one or more of the following unexpected and beneficial characteristics: high thermodynamic stability, chemical and physical stability in the solid state, excellent thermal properties (e.g., high melting point), high crystallinity (as shown by data presented herein), excellent impurity purging ability, low residual solvent content, and crystals that provide high flowability and ease of operation and manufacture. Accordingly, the crystalline forms according to the invention may be synthesized using a crystallization process that is more efficient and results in improved particle size and morphology relative to the corresponding amorphous and/or other forms of crystalline compound of Formula I. Employing a novel crystalline butenedioate Compound I, form 1 or crystalline Compound I, freebase, form 1 according to the invention may allow the use of conventional processing methods and formulation strategies. This is significant in that crystalline API (active pharmaceutical ingredient) forms typically have a reduced physical stability risk compared to the high energy state amorphous solid dispersions. Ultimately this may allow for less protective and potentially less expensive packaging configurations. A conventional formulation also allows for the use of standard, well-known processing trains (roller compaction, blending, and compression). These standard processing trains have been optimized to provide high yield, are easily scalable, and are abundant throughout the pharmaceutical manufacturing facilities worldwide. In addition, the manufacture of non-standard formulations of the compound of Formula I may require higher energy inputs (extrusion) or the use of solvents (e.g., spray drying). Thus, the novel crystalline forms according to the invention provide a potential for improved overall cost of goods.

Pharmaceutical Compositions

As noted above, another embodiment provides a pharmaceutical composition comprising a crystalline for according to the invention, e.g., crystalline anhydrous butenedioate Compound I, form 1 or anhydrous crystalline Compound I, free base, form 1. In such compositions, the crystalline form comprises either the sole active agent, or is optionally present in combination with one or more additional therapeutic agents. In either case, said pharmaceutical compositions can further comprise one or more pharmaceutically acceptable carriers, excepients and/or diluents. Non-limiting examples of additional therapeutic agents which may be useful in combination with a crystalline form of Formula I according to the invention include those selected from the group consisting of: (a) drugs that may be useful for the treatment of Alzheimer's disease and/or drugs that may be useful for treating one or more symptoms of Alzheimer's disease, (b) drugs that may be useful for inhibiting the synthesis Aβ, (c) drugs that may be useful for treating neurodegenerative diseases, and (d) drugs that may be useful for the treatment of type II diabetes and/or one or more symptoms or associated pathologies thereof

Oral administration of a pharmaceutically advantageous crystalline form of Formula I may surprisingly reduce the food effect, that is, oral administration of a formulation comprising a pharmaceutically acceptable crystalline form of Formula I may provide substantially the same compound I exposure and less variability in bioavailability across a patient population regardless of whether the formulation is administered under Fed Conditions or Fasted Conditions. Moreover, when the results of oral administration of a formulation comprising a pharmaceutically advantageous crystalline form of Formula I are compared to those obtained after administration of an equivalent amount of Formula I in the form of a conventional formulation, under either fed conditions or fasted conditions, the crystalline forms of Formula I may provide increased bioavailability, with lower variability in bioavailability across a population of subjects, and higher exposure levels (AUC) in healthy volunteers to whom it is administered. Moreover, it is believed that similar results are achieved in patients to whom formulation comprising a pharmaceutically advantageous crystalline form of Formula I is administered.

Exemplifying the invention is the use of the compounds disclosed in the Examples and herein. A pharmaceutically acceptable crystalline form of compound I is useful in a method of modulating muscarinic M1 receptor activity in a patient such as a mammal in need of such inhibition comprising the administration of an effective amount of the compound. The invention is directed to the use of a pharmaceutically acceptable crystalline form of compound I or a pharmaceutically acceptable salt thereof as modulators of muscarinic M1 receptor activity. In addition to primates, especially humans, it is possible that a variety of other mammals may be treated according to the method of the invention.

The subject treated in the methods of the invention is generally a mammal, such as a human being, male or female. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with the disorders with an effective amount of the compound of the invention.

As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a patient who is predisposed to such disease or disorder.

The terms “administration of” and or “administering a” compound should be understood to mean providing a compound of the invention or a prodrug of a compound of the invention to the individual in need thereof.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the invention encompass any composition made by admixing a pharmaceutically advantageous crystal of Formula I and a pharmaceutically acceptable carrier. By “pharmaceutically acceptable” it is meant the carrier, diluent or excipient must be compatible with the other ingredients of the formulation and not deleterious to the recipient thereof.

The scientific literature has implicated the M1 positive allosteric receptors in a wide range of biological functions. This has suggested a potential role for these receptors in a variety of disease processes in humans or other species. In accordance with the invention, a pharmaceutically advantageous crystalline salt of Formula I may have utility in treating, preventing, ameliorating, controlling or reducing the risk of Alzheimer's disease, schizophrenia, or disorders associated with M1 receptors. In accordance with the invention, a pharmaceutically acceptable crystalline form of compound I may provide methods for: preventing and treating Alzheimer's disease, schizophrenia, pain, or sleep disorders; in a mammalian patient in need thereof which comprises administering to the patient a therapeutically effective amount of a pharmaceutically acceptable crystalline form of compound I of the invention.

In accordance with the invention, a pharmaceutically advantageous crystalline form of Formula I may also be useful in a method for the prevention, treatment, control, amelioration, or reduction of risk of the diseases, disorders and conditions noted herein. The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.0001 to 10 mg/kg. of body weight daily are administered to the patient, e.g., humans and elderly humans, to obtain effective modulation of M1 receptors. The dosage range will generally be about 0.5 mg to 1.0 g. per patient per day which may be administered in single or multiple doses. In one embodiment, the dosage range will be about 0.5 mg to 500 mg per patient per day; in another embodiment about 0.5 mg to 200 mg per patient per day; and in yet another embodiment about 5 mg to 50 mg per patient per day. Pharmaceutical compositions of the invention may be provided in a solid dosage formulation such as comprising about 0.5 mg to 500 mg active ingredient, or comprising about 1 mg to 250 mg active ingredient. The pharmaceutical composition may be provided in a solid dosage formulation comprising about 1 mg, 5 mg, 10 mg, 15 mg, 20 mg, 25 mg, 30 mg, 40 mg, 50 mg, or 100 mg active ingredient. In specific embodiments, the pharmaceutical composition may be provided in a solid dosage formulation comprising about 10 mg, 15 mg, 20 mg, 30 mg, or 40 mg active ingredient. For oral administration, the compositions may be provided in the form of tablets containing 1.0 to 1000 milligrams of the active ingredient, such as 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compound may be administered on a regimen of 1 to 4 times per day, such as once or twice per day. In an embodiment of the invention, the compound may be administered in a regimen of once per day in the evening, such as prior to initiating sleep.

A pharmaceutically acceptable salt of crystalline Formula I that is employed in the invention may be used in combination with one or more other drugs in the treatment, prevention, control, amelioration, or reduction of risk of diseases or conditions for which compounds of the invention or the other drugs may have utility, where the combination of the drugs together are safer or more effective than either drug alone. Such other drug(s) may be administered, by a route and in an amount commonly used therefor, contemporaneously or sequentially with a compound of the invention. When a compound of the invention is used contemporaneously with one or more other drugs, a pharmaceutical composition in unit dosage form containing such other drugs and the compound of the invention is contemplated. However, the combination therapy may also include therapies in which the compound of the invention and one or more other drugs are administered on different overlapping schedules. It is also contemplated that when used in combination with one or more other active ingredients, the compounds of the invention and the other active ingredients may be used in lower doses than when each is used singly. Accordingly, the pharmaceutical compositions of the invention include those that contain one or more other active ingredients, in addition to a compound of the invention. The above combinations include combinations of a compound of the invention not only with one other active compound, but also with two or more other active compounds.

Likewise, the pharmaceutically advantageous crystalline form of Formula I thereof that is employed in the invention may be used in combination with other drugs that are used in the prevention, treatment, control, amelioration, or reduction of risk of the diseases or conditions for which compounds of the invention are useful. Such other drugs may be administered, by a route and in an amount commonly used therefor, contemporaneously or sequentially with a compound of the invention. When a pharmaceutically advantageous crystalline form of Formula I is used contemporaneously with one or more other drugs, a pharmaceutical composition containing such other drugs in addition to the compound of the invention is contemplated. Such pharmaceutical compositions are prepared without undue experimentation in accordance with the methods described herein and known in the art. Accordingly, the pharmaceutical compositions include those that also contain one or more other active ingredients, in addition to a compound of the invention.

The weight ratio of a pharmaceutically advantageous crystalline form of compound Ito the second active ingredient may be varied and will depend upon the effective dose of each ingredient. Generally, an effective dose of each will be used. Thus, for example, when a pharmaceutically advantageous crystalline form of Formula I is combined with another agent, the weight ratio of the compound of the pharmaceutically acceptable crystalline form of compound Ito the other agent will generally range from about 1000:1 to about 1:1000, such as about 200:1 to about 1:200. Combinations of a pharmaceutically acceptable crystalline form of compound I and other active ingredients will generally also be within the aforementioned range, but in each case, an effective dose of each active ingredient should be used. In such combinations the pharmaceutically advantageous crystalline form of Formula I and other active agents may be administered separately or in conjunction. In addition, the administration of one element may be prior to, concurrent to, or subsequent to the administration of other agent(s).

A pharmaceutical crystalline form of Formula I may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Pharmaceutical compositions intended for oral use may be prepared in accordance with the methods described herein and other method known to the art for the manufacture of pharmaceutical compositions. Such compositions may further contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions contain the active materials in admixture with excipients suitable for the manufacture of aqueous suspensions. Dispersible powders and granules suitable for preparation of an aqueous suspension by the addition of water provide the active ingredient in admixture with a dispersing or wetting agent, suspending agent and one or more preservatives. Formula I may be synthesized as described in WO 2010/059773, or in commonly owned, co-pending application under Attorney Docket No. Case 24194. Methods for preparing the crystalline compounds of this invention are illustrated herein. Starting materials are made according to procedures known in the art or as illustrated herein.

### EXAMPLE 1

### Preparation for Crystalline Freebase

A crude solution of Formula I free base in THF was concentrated to 2-3 volumes of THF, and 2-3 volumes of n-heptane were added to this solution along with seed. The batch was aged at 20-30° C. for 1-2 hours and an additional 2-3 volumes of n-heptane were added. The mixture was concentrated to 4-5 volumes and 3-4 volumes of heptane were added. The mixture was then concentrated to 4-5 volumes. The batch was then aged at 10-15° C. for 1-2 hours. The anhydrous crystalline free base form 1 of Compound I was then isolated by filtration.

### EXAMPLE 2

Preparation for Isolating Crystalline Butenedioate salt

Unseeded procedure: One molar equivalent of fumaric acid in water was added to a 9.4 mg/mL solution of crude Formula I free base in MeOH. The mixture was stirred at 50° C. for 2 hours. All solvent was then removed under reduced pressure. 0.4 mL of isopropyl acetate was added to the residual solids, and the mixture was stirred for 1 hour at 65° C. The mixture was then cooled to room temperature and birefringent solids were observed. Characterization of these solids, as described below, confirm that cyrstalline butenedioate salt form 1 of Compound I was isolated.

Seeded procedure: A 400 mg/mL solution of crude compound of Formula I free base in methanol was heated to 40° C. 1.05 molar equivalents fumaric acid was dissolved in 5.0 mL MeOH with heating to 40° C. 1 mL of fumaric acid solution was added to compound I freebase solution, and the mixture was aged 15 min at 40° C. Subsequently, 6.4 mg of butenedioate product was added as seed, inducing crystallization. The remaining fumaric acid solution was slowly added over one hour, followed by a 20 min age at 40° C. before adding 5 mL MeCN. Mixture was aged 10 min, 10 mL MeCN was added, and then aged another 10 min, then cooled slowly to RT and allowed to stir overnight. Solids were filtered and washed with 5 mL MeCN. Cyrstalline butenedioate salt form 1 of Compound I in the form of white solids were dried under N₂ and vacuum.

### EXAMPLE 3

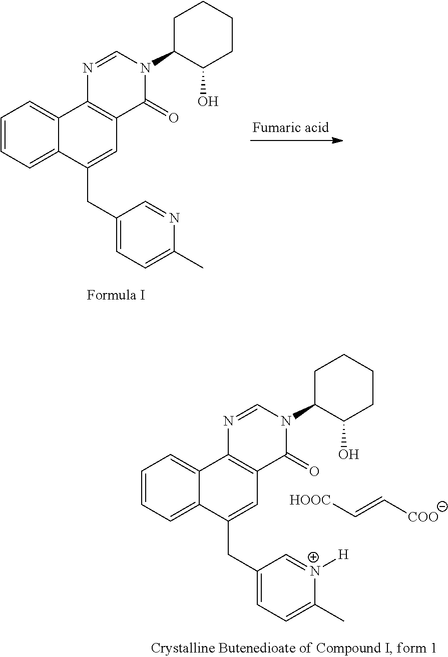

18.0 g of of the compound Formula I free base was dissolved in EtOH (270 mL) at 50° C. and filtered. In a separate vessel, fumaric acid (0.94 g) was dissolved in EtOH (32 mL) at 50° C. The fumaric acid solution was charged to the solution of via an in-line filter. The mixture was seeded with crystalline butenedioate of Compound I, form 1 (0.3 g) and the mixture was aged at 50° C. for 12 h.

In another vessel, additional fumaric acid (4.29 g) was dissolved in EtOH (130 mL) at 50° C. and then added to the above solution of Formula I free base and fumaric acid at 50° C. over 12 h. The combined batch was then cooled to 0° C. over 6 h and aged at 0 C for 4 h. The batch was then wet milled at 23-35 m/s tip speed for 2 hours. The milled batch was then heated to 50° C. for 1 h, then cooled to 0° C. over 3 h and aged at 0° C. for an additional 1 h. The batch was then filtered and washed with EtOH (52 L) to provide 20.57 g of crystalline butenedioate of Compound I, form 1 in 89% yield.

¹H NMR (d6-DMSO): 8.98 (1H, dd, J=7.6, 1.9 Hz), 8.64 (1H, br s), 8.44 (1H, d, J=2.0 Hz), 8.16 (1H, dd, J=7.2, 1.5 Hz), 7.93 (1H, s), 7.75 (2H, m) 7.47 (1H, dd, J=8.0, 2.3 Hz), 7.13 (1H, d, J=8.0 Hz), 6.63 (2H, s), 4.49 (3H), 4.03 (1H, br s), 2.40 (3H, s), 2.03 (1H, br s), 1.87-1.71(4H, br m), 1.36 (3H, br m).

### EXAMPLE 4

The two novel crystalline forms of Formula I described and characterized herein, surprisingly and advantageously, exhibit excellent biological and physical properties while minimizing the difficulties associated with drug substance manufacturing, processing and storage. For example, crystalline butenedioate of Compound I, form 1 has unexpectedly been found to be stable under forced stressed conditions as compared to the corresponding amorphous HCl salt. Forced stress experiments were performed using the procedures described in the WAT Guideline for Early Phase Analytical Method Development which is found in the FDA November 2003 ICH guidelines for stability testing.

The hydrogen peroxide stress (2-electron oxidation) experiment was evaluated using HPLC (Agilent Zorbax Eclipse Plus C18 600 Bar (4.6×50 mm)) to monitor degradation formation for up to 24 hours of a solution of crystalline butenedioate Compound I, form 1, or amorphous HCl salt diluted to volume using the 3% hydrogen peroxide solution (3% hydrogen peroxide in 50:50 methanol:water) that had been mixed well and stored at room / ambient temperature. Under these conditions crystalline butenedioate of Compound I, form 1 in the presence of 3% hydrogen peroxide showed less than 0.1% degradation at ambient and 5° C. temperatures. By contrast, the amorphous HCl salt was most sensitive to 3% hydrogen peroxide and basic conditions. There was 48% degradation in the presence of H₂O₂ and similar percentage of degradation was seen at both ambient and 5° C. temperatures.

The base catalyzed hydrolysy experiment was conducted using HPLC (Agilent Zorbax Eclipse Plus C18 600 Bar (4.6×50 mm)) to monitor loss of active/degradate formation for up to 3 days of a solution of crystalline butenedioate Compound I, form 1, or amorphous HCl salt that had been diluted to volume using 1N NaOH, mixed well. The experiment was conducted on a sample stored at room/ambient temperature and another sample maintained at 60° C. Under basic conditions (0.1 N) crystalline butenedioate of Compound I, form I showed a degradation of 1.4% after 24 hours at 60° C. and only 0.2% at ambient temperature. This degradation didn't increase after two more days at the study conditions. On the other hand, under basic conditions (0.1 N) with amorphous HCl salt there was approximately 39% loss at 24 hr and ambient temperature and this increased to 64% loss at 60° C.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

20. Anhydrous crystalline compound I butenedioate salt form 1 represented by structural formula:

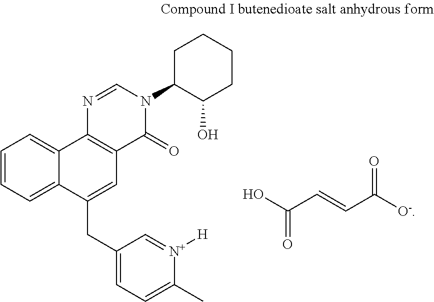

21. The compound of claim 20 characterized by an x-ray powder diffraction pattern, collected using copper K-alpha radiation, corresponding to d-spacings of 18.78, 12.81, 9.24, 8.97, 8.09, 6.84, 6.40, 5.83, 5.71, 5.45, 5.09, 5.04, 4.86, 4.73, 4.63, 4.58, 4.52, 4.38, 4.19, 4.17, 4.06, 3.93, 3.84, 3.77, 3.70, 3.64, 3.55, 3.42, 3.37, 3.26, 3.20, 3.12, 3.05, 3.01, 2.97, 2.87, 2.82, 2.79, 2.76, 2.71, 2.67, 2.62. 2.59, 2.54, 2.47, 2.45, 2.43, 2.39, 2.34, and 2.32 angstroms.

22. The compound of claim 20 characterized by an x-ray powder diffraction pattern, collected using copper K-alpha radiation, corresponding to d-spacings of 12.81, 9.24, 8.97, 6.40, 5.83, 5.71, 5.09, 4.63, 4.58, 4.52, 4.38, 3.93, 3.84, 3.77, 3.70, 3.64, 3.55, 3.42, 3.37, 3.20, and 3.12angstroms.

23. The compound of claim 20 characterized by an x-ray powder diffraction pattern, collected using copper K-alpha radiation, corresponding to d-spacings of 12.81, 8.97, 6.40, 5.83, 5.71, 4.63, 4.58, 4.52, 4.38, 3.77, 3.64, 3.55 and 3.37 angstroms.

24. The compound of claim 23 characterized by an x-ray powder diffraction pattern, collected using copper K-alpha radiation, corresponding to d-spacings of 12.81, 4.52, 4.38, 3.55 and 3.37 angstroms.

25. Anhydrous crystalline freebase form 1 of Compound I represented by structural formula:

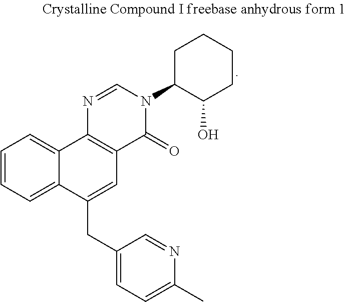 [SMILES: Cc1ccc(Cc2cc3c(=O)n([C@H]4CCCC[C@@H]4O)cnc3c3ccccc23)cn1]

26. The compound of claim 25 characterized by an x-ray powder diffraction pattern, collected using copper K-alpha radiation, corresponding to d-spacings of 19.83, 9.92, 9.09, 8.09, 7.10, 6.58, 6.25, 5.53, 5.12, 5.07, 4.94, 4.77, 4.58, 4.44, 4.29, 4.16, 4.03, 3.89, 3.80, 3.71, 3.55, 3.48, 3.43, 3.37, 3.33, 3.29, 3.19, 3.12, 3.08, 3.00, 2.97, 2.91, 2.85, 2.73, 2.67, 2.55, 2.50, 2.46, 2.39, and 2.33angstroms.

27. The compound of claim 25 characterized by an x-ray powder diffraction pattern, collected using copper K-alpha radiation, corresponding to d-spacings of 19.83, 9.92, 9.09, 6.25, 5.53, 5.12, 5.07, 4.94, 4.77, 4.58, 4.44, 4.29, 4.03, 3.80, 3.71, 3.48, 3.43, 3.37, 3.33, 3.00, 2.73, and 2.55 angstroms.

28. The compound of claim 25 characterized by an x-ray powder diffraction pattern, collected using copper K-alpha radiation, corresponding to d-spacings of 19.83, 9.92, 9.09, 5.53, 5.12, 5.07, 4.77, 4.44, 4.29, and 3.43 angstroms.

29. The compound of claim 25 characterized by an x-ray powder diffraction pattern, collected using copper K-alpha radiation, corresponding to d-spacings of 9.92, 5.12, and 5.07 angstroms.

30. A pharmaceutical composition comprising the compound of claim 20 and a pharmaceutically acceptable carrier.

31. A pharmaceutical composition comprising the compound of claim 25 and a pharmaceutically acceptable carrier.

32. A method for treating Alzheimer's disease in a human patient in need thereof which comprises administering to the patient a therapeutically effective amount of the compound of claim 20.

33. A method for treating Alzheimer's disease in a human patient in need thereof which comprises administering to the patient a therapeutically effective amount of the compound of claim 25.

34. A method for treating diseases mediated by muscarinic M1 receptors in a human patient in need thereof which comprises administering to the patient a therapeutically effective amount of the compound of claim 20.

35. A method for treating diseases mediated by muscarinic M1 receptors in a human patient in need thereof which comprises administering to the patient a therapeutically effective amount of the compound of claim 25.

36. A process process for making a crystalline compound of Formula I represented by structural formula: 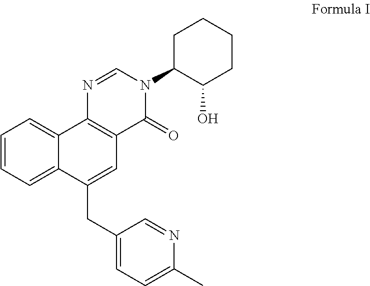 [SMILES: Cc1ccc(Cc2cc3c(=O)n([C@H]4CCCC[C@@H]4O)cnc3c3ccccc23)cn1]
comprising contacting the compound of Formula I, free base with an acid in the presence of an organic solvent, adding Compound I butenedioate form 1 as a seed and aging the solution for up to 48 hours at a temperature of about 25 C to about 85 C, cooling the solution to a temperature of about −20 C to about 15 C and isolating anhydrous crystalline compound of Formula I.

37. The process according to claim 35 xs wherein the acid is selected from the group consisting of acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methansulfonic, mucic, nitric, pamoic, pantothenic, phosphoric, succinic, sulfuric, tartaric, and p-toluenesulfonic acid, and the organic solvent is selected from the group consisting of EtOH, MeOH, iPrOH, CH₂Cl₂, THF, 2-MeTHF, MTBE, DME, 1,4-dioxane, CPME, EtOAc, iPrOAc, tBuOH, t-AmOH, toluene, DMF, DMAc, NMP, and DMSO.

38. The process according to claim 35 wherein the acid is fumaric acid, organic solvent is EtOH, and the solution is cooled to a temperature of about 0° C. to about 5° C. at a rate of about 5° C. per hour and aged at a temperature of about 0° C. to about 5° C. for up to 24 hours resulting in crystalline butenedioate Compound I, form 1 of structural formula : 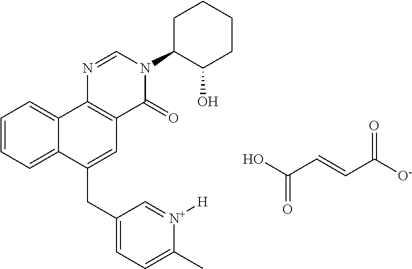 [SMILES: Cc1ccc(Cc2cc3c(=O)n([C@H]4CCCC[C@@H]4O)cnc3c3ccccc23)c[nH+]1 ; O=C([O-])/C=C/C(=O)O]
